(19) United States
(12) Patent Application Publication
(10) Pub. No.: US 2008/0027329 A1
(43) Pub. Date: Jan. 31, 2008

(54) System, apparatus and method for measurement of motion parameters of an in-vivo device

(76) Inventor: Arkady Glukhovsky, Santa Clarita, CA (US)

Correspondence Address: PEARL COHEN ZEDEK LATZER, LLP, 1500 BROADWAY 12TH FLOOR, NEW YORK, NY, 10036, US

(21) Appl. No.: 11/822,776

(22) Filed: Jul. 10, 2007

Related U.S. Application Data
(62) Division of application No. 10/928,260, filed on Aug. 30, 2004.
(60) Provisional application No. 60/498,594, filed on Aug. 29, 2003.

(51) Int. Cl. A61B 6/00

(52) U.S. Cl. 600/476

(57) ABSTRACT

A system, apparatus and method may measure motion parameters of an in-vivo device, utilizing for example surface or flow irregularities for calculation of movement, distance, velocity etc. There may be provided with an in-vivo imaging device a motion parameter measurement unit that may include one or more of illumination sources and a plurality of illumination detectors located on an in-vivo device, such as a swallowable capsule.

### RELATED APPLICATION DATA

This application claims benefit from U.S. provisional application Ser. No. 60/498,594, filed on Aug. 29, 2003, entitled SYSTEM, APPARATUS AND METHOD FOR MEASUREMENT OF MOTION PARAMETERS OF AN IN-VIVO DEVICE which is incorporated in its entirety by reference herein.

### FIELD OF THE INVENTION

The present invention relates to systems, apparatuses and methods useful for in-vivo imaging. Specifically, embodiments of the present invention relate to systems and methods that enable measuring of motion parameters for in-vivo imaging devices.

### BACKGROUND OF THE INVENTION

Devices helpful in providing in-vivo imaging are known in the art. Autonomous in-vivo imaging devices, such as swallowable capsules, may move through or around a body lumen or a cavity. However, data acquired by the in-vivo imaging device may not relate to the motion parameters of the in-vivo imaging device, such as distance traveled, velocity of propulsion, peristaltic waves etc., at the time that images were acquired.

There are various reasons why it may be important to know the motion parameters of such an autonomous in-vivo imaging device in or along a body lumen, such as the gastrointestinal tract. However, since a typical in-vivo imaging device may sometimes move forwards, backwards, back and forth and/or travel non-uniformly inside a body lumen, it may be difficult for an operator of such an in-vivo imaging device to determine the motion parameters of the in-vivo imaging device when the various images are acquired. Furthermore, movements of the body lumen, besides movements of the in-vivo imaging device, may further impact on the path length traversed by a capsule.

It would be advantageous to have a system, apparatus and/or method for accurately determining motion parameters related to the movement of an in-vivo imaging device.

### SUMMARY OF THE INVENTION

There is provided, in accordance with some embodiments of the present invention, an apparatus, system, and method for enabling determination of motion parameters of an in-vivo imaging device, such as path length traversed, instant and average velocity of propulsion, frequency of peristaltic waves, or other relevant motion parameters. Such embodiments may utilize, for example, surface or flow irregularities for calculation of movement parameters, distance, and flow etc. According to one embodiment of the invention there may be provided, in an in vivo device at least one imaging apparatus, one or more energy sources such as motion dedicated illumination sources and a plurality of motion dedicated illumination detectors.

According to one embodiment the illumination sources may illuminate a lumen or cavity wall and the detectors may measure different reflections or energy receptions from the wall, at different locations along the wall, due, for example, to observable irregularities of the wall. While an in-vivo device, such as, for example, an autonomous capsule, moves along a lumen illuminating the lumen wall, the output of the various detectors may fluctuate. Analysis of the output of the detectors, together with, for example, information regarding the typically fixed distance between the detectors, may enable measurement of the relative movement (backward or forward etc.), distance and speed etc. of the in-vivo imaging device. The path length traversed by the in-vivo imaging device may be also calculated.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic illustration of components of an in-vivo imaging system, according to some embodiments of the present invention;

FIG. 2A is a schematic illustration of an in-vivo device, according to some embodiments of the present invention, wherein two motion illumination sources and two motion illumination detectors have been integrated;

FIG. 2B is a schematic illustration of an in-vivo device, according to some embodiments of the present invention, wherein a mirror for imaging has been integrated;

FIGS. 3A, 3B and 3C illustrate graphs representing in-vivo imaging device motion parameters, according to some embodiments of the present invention;

FIG. 4 is a flow chart describing a workflow for calculating motion parameters of an in-vivo device, according to an embodiment of the present invention;

FIG. 5 is a schematic illustration of multiple motion illumination sources and detectors applied to an in-vivo device, according to some embodiments of the present invention;

FIG. 6 is a schematic illustration of an in-vivo device that includes at least one motion illumination source and at least one motion imager, according to some embodiments of the present invention; and

FIG. 7 is a graphic illusion denoting an estimated path taken by an in-vivo device, according to an embodiment of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements throughout the serial views.

### DETAILED DESCRIPTION OF THE INVENTION

The following description is presented to enable one of ordinary skill the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the following detailed description, numerous specific details are set forth in order to provide a thorough understanding of the present invention. However, it will be understood by those skilled in the art that the present invention may be practiced without these specific details. In other instances, well-known methods, procedures, and components have not been described in detail so as not to obscure the present invention.

Unless specifically stated otherwise, as apparent from the following discussions, it is appreciated that throughout the specification discussions utilizing terms such as “processing”, “measuring”, “computing”, “calculating”, “determining”, or the like, may refer to the action and/or processes of a processor, microprocessor, “computer on a chip”, computer, workstation or computing system, or similar electronic computing device, that manipulate and/or transform data represented as physical, such as electronic, quantities within the computing system's registers and/or memories into other data similarly represented as physical qualities within the computing system's memories, registers or other such information storage, transmission or display devices.

Embodiments of the present invention may enable apparatuses, systems, and methods for measuring at least one motion parameter (e.g., distance traveled, velocity, acceleration, etc.). The system and method may use, for example surface or flow irregularities for calculation of movement parameters, distance and flow etc. According to some embodiments measurement of local electrical or mechanical properties such as electrical and acoustic impedances may be utilized to enable calculation of motion parameters.

There are various reasons why it may be important to know the motion parameters of such an autonomous in-vivo device in or along a body lumen, such as the gastrointestinal tract. Knowledge of motion parameters may enable physicians skilled in the art to provide, for example, improved diagnostic capabilities of the device by improved identification of the examined organ; indications of feasibility of subsequent endoscopic treatment by showing whether the identified pathology is within or out of the reachable range; automatic delivery and releasing of drugs (or other treatments) after passing a pre-defined distance along the body lumen; calculations of motility (position changes, motion etc.), for example, net propulsion of an in-vivo imaging device, and instant and average velocity of propulsion; calculations of peristaltic wave frequency; and improvements in image dilution and image stitching etc. Of course, other uses and benefits of such information are possible, and are within the scope of the present invention.

Some embodiments of the present invention are directed to a typically swallowable in-vivo device, such as an autonomous swallowable capsule. Other embodiments need not be swallowable or autonomous, and may have other shapes or configurations. Devices according to embodiments of the present invention, including imaging, receiving, processing, storage and/or display units suitable for use with embodiments of the present invention, may be similar to embodiments described in International Application WO 01/65995 and/or in U.S. Pat. No. 5,604,531, each of which are assigned to the common assignee of the present invention and each of which are hereby incorporated by reference. Of course, devices and systems as described herein may have other configurations and other sets of components.

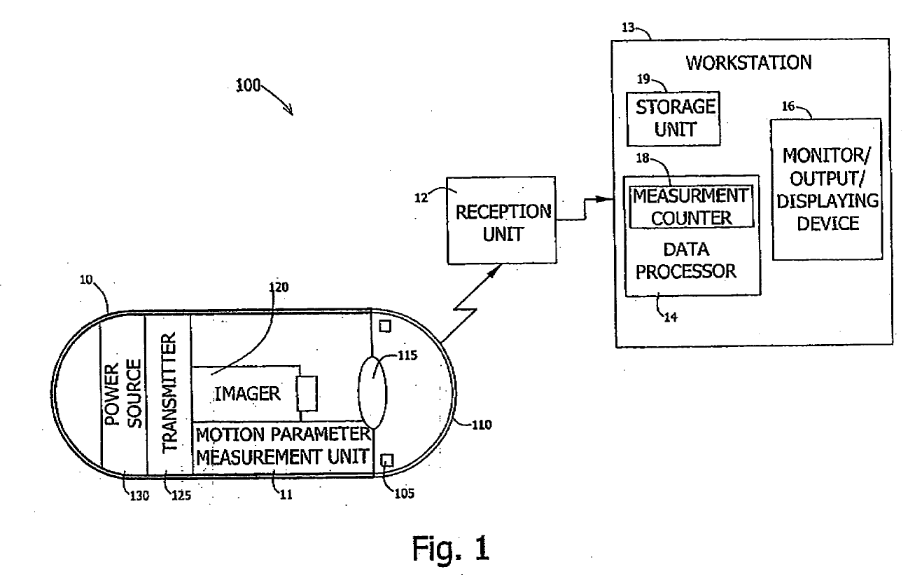

Reference is now made to FIG. 1, which illustrates components of an in-vivo imaging system 100, according to some embodiments of the present invention. As can be seen in FIG. 1, an in-vivo device 10, such as a swallowable capsule, may be provided with an imaging dedicated illumination source 105; a viewing window 110 through which the inner portions of body lumen or cavities, such as the GI tract, may be illuminated; a camera or imaging system, including an imager 120, such as a CMOS imager, which may detect images; an optical system 115, typically including a lens, which may focus the images onto the imager 120; a transmitter 125 which may transmit signals from the imager 120; and a power source 130, such as a battery, which provides power to the electrical elements of device 10.

Transmitter 125 includes control capability for, for example controlling the various operations of device 10, although control capability or one or more aspects of control may be included in a separate component. Transmitter 125 is typically an ASIC (application specific integrated circuit), but may be of other constructions; for example, transmitter 125 may be a processor executing instructions. Device 10 may include a processing unit separate from transmitter 125 that may, for example, contain or process instructions.

In one embodiment, all of the components may be sealed within the device body (the body or shell may include more than one piece); for example, an imager 120, illumination sources 105, power source 130, and transmitter 125 and control unit may all be sealed within the device 10 body.

A motion parameter measurement unit 11 may also be provided, to detect and/or enable determination of in-vivo imaging device motion parameters. According to one embodiment the motion parameter measurement unit 11 may reside externally to the device body, for example, in an extra-body unit. Other components or sets of components may be used. For example, a charge-coupled device (CCD) camera or any other suitable imaging device(s) may be used, and other power sources (such as external power sources) may be used.

A reception unit 12 may be provided for receiving in-vivo device data. A data processor 14 may be provided for processing data. An output device 16, such as a monitor or other suitable data displaying apparatus, which may display output data, such as image data or other data. For example, a reception unit 12 may receive data from in-vivo device 10, and may thereafter transfer the data to data processor 14, and/or to data storage unit 19. The recorded data and/or processed data may be displayed on a displaying device 16 or any other suitable output device. A measurement counter 18 for measuring selected times or time periods of in-vivo imaging device functioning may be provided within reception unit 12. Alternatively, measurement counter 18 may be located within in-vivo device 10, within data processor 14, or in any other suitable location. According to alternate embodiments the motion parameter measurement unit 11 may include a counter and may be located in the reception unit 12 or in the workstation 13.

Reception unit 12 may be separate from processing unit 14 or may be included within it. Data processor 14 may be included within, for example, a computer system or workstation 13, and may include, for example, a processor, memory, software code etc. Data processor 14 may be configured for real time processing and/or for post processing of in-vivo imaging device 10 data, including, for example, analyzing, calculating, viewing, displaying or otherwise implementing any other suitable functions relevant to such data and/or the in-vivo imaging device motion parameters derived therefrom. Alternately, certain data processing capability and a data processor unit, or at least a part of a data processor 14, may be incorporated into in-vivo device 10, for example, within an Application Specific Integrated Circuit (ASIC) that may be located within transmitter 125 or imager 120, or any other in-vivo device component. One or more of units 12, 14, 15, 16, and 19 may be integrated into a single unit, such as workstation 13, or may be integrated into reception unit 12, or any other suitable component. Any combinations of the various units may be provided. Of course, other suitable components may be used.

The location, movement, path length etc. of device 10 may be displayed in various suitable manners on, for example, displaying device 16. While in some embodiments the structure and functioning of the receiving, processing and display systems or units are similar to those described in U.S. Pat. No. 5,604,531 and/or International Patent Number WO 01/65995, other structures, functionalities and methods may be used.

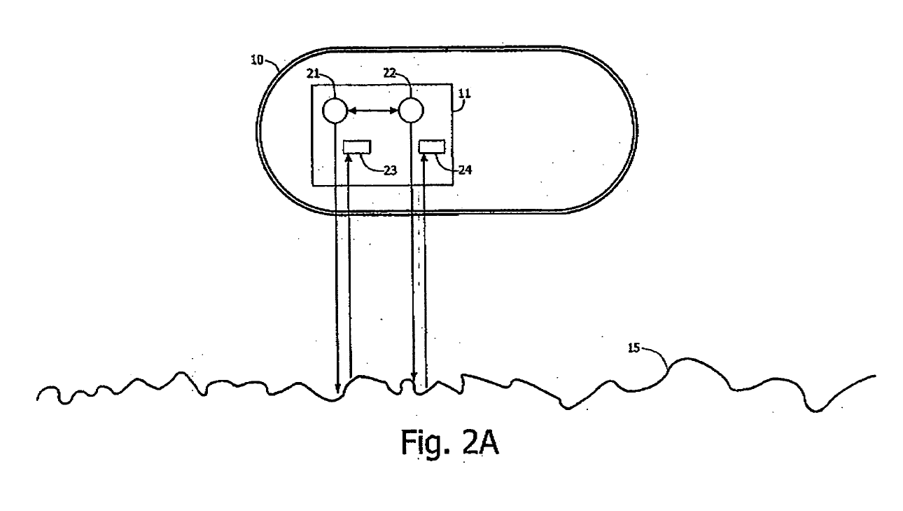

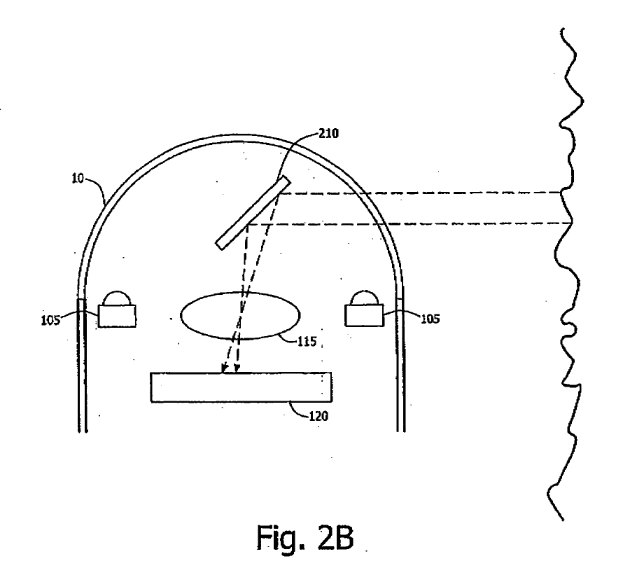

Reference is now made to FIG. 2A, which illustrates an example of an in-vivo device 10, including motion parameter measurement unit 11, according to some embodiments of the present invention. One embodiment of motion parameter measurement unit 11 includes a set of energy output units or sources such as motion dedicated illumination sources 21 and 22 (also referred to as motion illumination sources), and a set of motion dedicated illumination detectors 23 and 24 (also referred to as motion illumination detectors), for enabling determination of motion parameters of in-vivo device 10. While in one embodiment each source is paired with a detector, in other embodiments this need not be the case. Typically, the detectors are configured so that as the device moves, generally the same region is detected via reflection or energy reception. For example, the detectors may be arranged in a line along the expected direction of movement of the device. Motion illumination sources 21 and 22 may be any suitable light source, such as, LEDs, incandescent lamps, or alternate illumination apparatuses that may provide visible illumination, infrared illumination, ultraviolet illumination, and/or other suitable illumination types. Motion illumination detectors 23 and 24 may be sensor device, for example, optical sensing devices such as photodiodes, imagers or any other suitable imaging devices. According to some embodiments motion illumination detectors 23 and 24 may be sensor devices, for example, electrical impedance measurement devices such as pairs of electrodes or any other suitable measurement devices. Motion illumination detectors 23 and 24 way be capable of receiving and/or recording reflected or received energy such as illumination, including visible illumination, infrared illumination, ultraviolet illumination, and/or other suitable illumination types.

Other types of energy output units or sources, other than illumination sources 21 and 22, may be used. For example, one or more energy sources outputting, for example, acoustic energy or electric energy may be used; if so, corresponding appropriate energy receiving units (e.g., electrodes, acoustic detectors, etc.) may be used. While in one embodiment the energy producing units and energy receiving units are paired (e.g., source 21 is paired with detector 23), in other embodiments, such pairings need not be used. For example, one energy unit such as an illumination source may provide illumination, and a set of detectors placed an appropriate distance apart may receive reflection data.

In FIG. 2A, two motion illumination sources 21 and 22 are depicted, but other numbers may be used. Certain components depicted in FIG. 1, such as imager 120, illumination source 105, etc., are not depicted in FIG. 2A for the sake of clarity. Motion illumination sources 21 and 22 are typically separate from the image illumination sources 105 shown in FIG. 1, but need not be, and may be used for lumen illumination and/or motion detection. Illumination sources 105 of in-vivo device 10 (FIG. 1) may function as motion illumination sources 21 and 22. Motion illumination detectors 23 and 24 are typically separate from imager 120, but need not be. According to some embodiments imager 120 may function as a motion illumination detector. An additional embodiment using a similar imager 120 and similar illumination sources 105 for both imaging and for detection of motion parameters is shown if FIG. 2B. In this embodiment a mirror 210 for inking may be integrated into in-vivo imaging device 10.

Motion illumination detectors 23 and 24 may be integrated within an in-vivo device 10, typically on at least one side of in-vivo device 10 but optionally in other locations. Each motion illumination source 21, 22 may periodically or continually illuminate a point along a lumen wall 15, the reflection of which may be recorded by the relevant motion illumination detectors 23 and/or 24. Such illumination is typically simultaneous, but need not be. Motion illumination sources 21 and 22, and motion illumination detectors 23 and 24 may include, for example, laser diodes, regular lenses or micro-lenses which may be attached to diodes/detectors, to enable generation and/or reception of point-wise illumination. In some embodiments an array of motion illumination sources may be provided, positioned on the side(s) and/or other locations of the circumference of the in-vivo device 10. In some embodiments a single motion illumination source may be provided.

During a time period (T_{window}), detectors 23 and 24 may periodically or constantly measure reflections or reception of energy such as for example illumination generated by motion illumination sources, such as 21 and 22. The data representing the illumination received by the various detectors may be transferred, by transmitter 125, to a processor or controller unit, such as, for example, data processor 14. Alternatively, the processor or controller unit may be located within in-vivo device 10, such as, for example, within transmitter 125 or imager 120. This data may include for example image data of the reflection recorded by an illumination detector, such as imager 120, or detectors 23 and 24, the time at which the image was recorded, as well as any other related data, such as intensity, hue, and/or color. The time may be derived from, for example, an in-vivo device 10 master clock, which may be integrated into, for example, an ASIC as part of transmitter 125, reaction unit 12, or any other component in in-vivo imaging system 100. In other embodiments, the time need not be transmitted. A description of an example of a master clock may be seen in the above-mentioned patent application, WO 01/65995. The data may be received by reception unit 12, or may be transferred directly to data processor 14. In addition to an image frame there may be a header which may include various telemetry data, such as temperature, pH, pressure, etc. Motion parameter data may be, for example, recorded by motion illumination detectors 23 and/or 24, and transmitted as part of the header to reception unit 12, storage unit 19 and/or workstation 13. Alternatively, motion parameter data may be, for example, transmitted as part of an image data frame. For example, pixels from the corner of the image, which may be unused or less important, may be substituted by the motion parameter data. In one embodiment, collected reflection or reception data from different detectors may be compared, and the movement or movement parameters of the device determined. For example, one or more illumination signals may be output. Two detectors, a known distance apart, may record the signals. Typically, the detectors are configured so that as the device moves, generally the same region is detected via reflection. The signals may be compared by, for example, a cross-correlation function, to determine movement parameters.

Due to surface or flow irregularities etc. typical of body lumen walls 15 or cavities, motion illumination detectors 23 and 24 may measure different reflections at different locations along lumen wall 15. Each of the different locations that are imaged, for example, by motion illumination detector 24 may function as markers or flags by which to determine whether, for example, motion illumination detector 23 acquired data from the same location. For example, the reflection acquired by illumination detector 23 may be more or less intense, colorful or graded etc., than the reflection of illumination source 24, indicating different depths, colors or alternative surface characteristics of the recorded illumination at the two points. In this way, when illumination detector 23 acquires data at the same location or spot previously detected (e.g., imaged) by illumination detector 24, processor 14 (or any other processing unit) may identify that a similar location has been acquired by both illumination detectors 23 and 24, and thereafter calculate the time difference in detecting of this location by the two illumination detectors 23 and 24. Since the distance between the locations of the two illumination detectors 23 and 24 on in-vivo device 10 is typically fixed, the distance traversed by in-vivo device 10 may be determined, and the velocity, direction of movement and various other movement parameters of in-vivo device 10 may be calculated.

While device 10 moves along or within a lumen, the output of both detectors 23, 24 may typically fluctuate. The output from each detector 23 and 24 may be assigned a time using, for example, a clock in device 10 or in an external recording and/or receiving system. Thus each data point recorded by detector 23 and 24 may be stored with or associated with a time. The time may be an absolute time or a relative time (e.g., since the recording started). Alternately, data points may be associated with a value other than time, such as frame number, etc. When performing distance calculations, such values may be converted to time values, but need not be. A function (e.g., a graph) may be calculated or plotted for the movement in time of each detector 23 and 24.

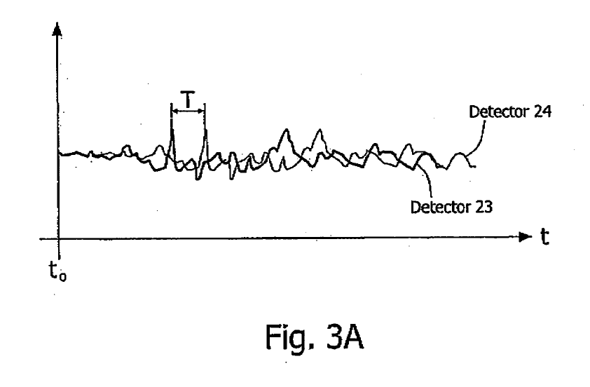

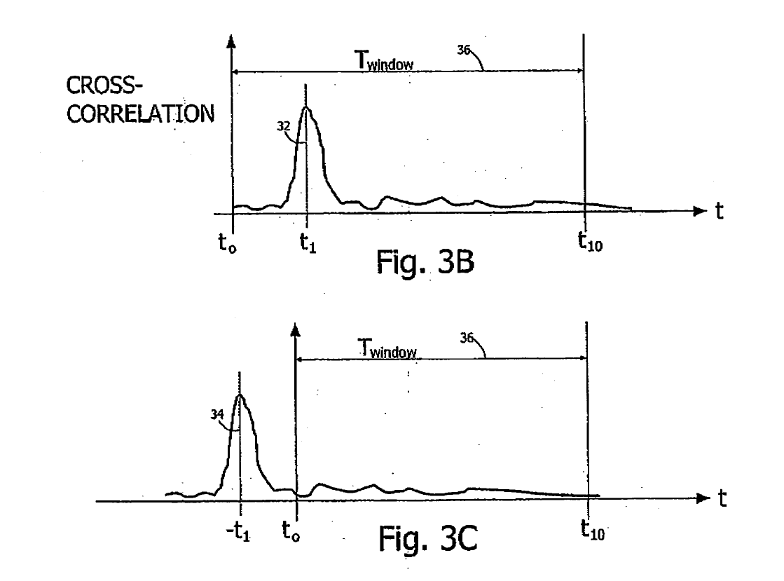

An example of a fluctuation between the recorded reflections of illumination detectors 23 and 24 may be seen in the chart illustrated in FIG. 3A. The graph in FIG. 3A illustrates the relative illumination detector outputs, for example of units of illumination intensity, hue, and/or color etc., for detectors 23 and 24, at a plurality of points in time (t). The two functions of the respective detectors, as illustrated by the solid and dotted lines respectively in FIG. 3A, may be similar in shape because they are a reflection of similar wall irregularities, as recorded by illumination detectors 23 and 24. Other suitable methods of representing and/or processing data from detectors 23 and 24 may of course be used. The data sets collected by the two detectors 23 and 24 may be compared to determine the movement of device 10. In one embodiment, the comparison may be performed between or among graphs created by data points from detectors 23 and 24, using, for example, cross-correlation functions. Other suitable comparisons are within the scope of the present invention.

Since illumination detectors 23 and 24 are located at different places on in-vivo device 10, and therefore the second detector 23 may record reflected illumination at time interval “T” after that of the first detector 24, the two functions or graphs based on collected data may be shifted or separated in time by “T” for each recorded frame. As can be seen in FIG. 3A, intensity of the reflection may be seen for two illumination detectors 23 and 24. “T” is the time shift between the output of detector 24 and detector 23, and may therefore be defined as the time that it took the in-vivo imaging device to move distance “D”, which is the displacement between detectors 23 and 24 (FIG. 2). It may therefore be expected, that if the in-vivo device 10 were to be moving with constant motion parameters, the two functions would have similar shapes, and be shifted by time (T). Since the second detector 24 may not be moving along exactly the same path of the first sensor, for example, due to in-vivo device 10 rotation or lumen movement etc., the functions of the two detectors may be different. Any other illumination parameters may similarly be measured, for example, illumination intensity, color, hue, etc., or any other parameters that may produce a graph of difference over time, based on analysis of the respective functions.

The output from the detectors may be displayed. Outputs may be provided for a plurality of periods and/or a plurality of detectors. Outputs may indicate in-vivo device 10 motion parameters, such as movement, direction and path length traversed etc. The shift or difference between the curves may vary depending on, for example, the velocity of the device 10. Furthermore, due to variations in detections, rotation or lateral movement of the device 10, the curves or graphs may not precisely match.

Various cross-correlation analyses, for example, may be performed on the respective functions or collections of data, representing the output of respective detectors during some pre-defined period(s) of time. “T_{window}” 36, is depicted in FIGS. 3B and 3C as the period of time between t₀ and t₁. T_{window} may define the length of a function, for example, the output of the detectors 23 and 24 during a selected time interval. T_{window} may preferably be selected to include a time interval during which the in-vivo imaging device motion parameters do not change significantly. Any length T_{window} periods may be selected, and any number of T_{window} periods may be used. If T_{window} is too short, there may be a small number of data points, and the cross-correlation function may be inconclusive. If the T_{window} is too long, it may lead to factoring in to the time comparison additional motion parameters, for example, the time period may be substantially large so as to allow for substantial movement of the lumen or cavity wall, rotation of the capsule etc. For example, a “too short” period can be defined by the resulting Signal/Noise ratio of the cross correlation function, and may be determined for each process. In the case of movement in the intestine this time may be in the order of several milliseconds. A “too long” period can be determined by duration of movement with the same parameters, for example, in the order of hundreds of milliseconds. The cross-correlation analyses, which may typically be performed by processor 14, but may be performed by other processors or controllers, may enable calculation of time required for one detector to move to the location of the other detector. In order to detect rotation, an array of detectors may be placed along the in-vivo imaging device circumference, such that cross-correlation techniques may be implemented on at least a part of the two dimensional array of detectors. According to some embodiments a three-dimensional array may be used. Functions or processing other than cross-correlation functions may be used. Output of the cross correlation between two functions may be a function, and may be expressed in units of distance, velocity, rotational degrees etc. A maximum output from the cross-correlation may occur in the time that corresponds to the shift in time between the two functions.

A cross-correlation analysis implemented, for example, on a pair of detector outputs separated by time “T” may result, for example, in a function having a distinctive peak at time “t₁”, as can be seen in FIGS. 3B and 3C. This peak, such as the peak near line 32, for example, when appearing in relation to two different data sets or functions, may represent, the time that it took the capsule to move the distance between the two sensors. The resulting function may be used to calculate or otherwise determine, for example, the time it takes for the in-vivo device 10 to travel the distance between the two detectors. Methods other than cross correlation functions may be used to compare two or more sets of data. Once T is known, and the distance (D) between the respective detectors is known, various motion parameters may be calculated or determined, for example in-vivo events or changes, distance traversed, velocity of movement, rotating, reversing or otherwise altering of in-vivo imaging device motion parameters etc. FIGS. 3B and 3C indicate, for example, the distance moved by in-vivo imaging device 10 during time period T_{window}. As can be seen, in FIG. 3B in-vivo device 10 moved forward during the window period, as indicated by line 32. In FIG. 3C in-vivo device 10 moved backwards, as indicated by line 34. The degree of backward or forwards movement, for example, may be analyzed, optionally in relation to analyses of additional window periods, to provide additional movement parameters. The above results may be attained, for example, by calculating various cross correlation functions between data corresponding to the outputs of detector 23 and detector 24.

By analyzing the output of detectors 23 and 24, knowing the distance between the detectors (which is typically constant, but may not be), and optionally using the time parameters which may, for example, be provided by a master clock, processor 14 or another suitable processor may calculate various motion parameters, such as relative movement (backward or forward), distance and speed etc. of an autonomous in-vivo device, such as device 10. For example, once the cross correlation function is calculated, the time required to the second sensor to get to the location of the first sensor, which is time period “T”, may be known. Since the distance between the sensors (distance “D” in FIG. 2) is also known, the instant velocity of in-vivo device 10 may be calculated, by using, for example, the formula: V=D/T. It should be noted that cross-correlation functions may provide negative results, such as in the case where device 10 moves backwards. For example, the velocity at various points, the velocity displayed with each of a series of images displayed, and other movement parameters may also be calculated.

According to some embodiments of the present invention any suitable dimensions may be used for in-vivo device 10, and, for example, between motion illumination detectors 23 and 24. In one embodiment the distance between the motion illumination detectors 23 and 24 may be, for example, 0.1-20 mm apart. The dimensions of device in-vivo device 10 may be, for example, length 25 mm and diameter of 11 mm. The in-vivo device 10 motion parameters may be displayed in various suitable manners on various suitable displaying devices 16, such as monitors. Motion parameters may be displayed in graphs, maps, images, charts, video, or any other suitable forms. Other suitable dimensions may be used.

According to some embodiments of the present invention, an estimated path length of the in-vivo device 10 path may be also calculated. The instant and/or average velocity of propulsion, and frequency of peristaltic waves etc. may also be calculated. One embodiment of the present invention may include a method for calculation of the estimated path length. For example, when the first detector 23 arrived at location X this may be identified. When the second detector 24 arrived at location X this may be identified. The instant velocity of movement of in-vivo device 10 at location X, for example, may be calculated using the fixed distance between illumination detectors 23 and 24. Instant velocity at location X may be calculated, for example, according to a function such as V(t)=D/T(t). The above process may be repeated for a plurality of locations and way enable estimation of the path traversed by in-vivo device 10, or other motion parameters of in-vivo device 10. Further, other motion parameters, for example, an average velocity during any time interval may be calculated in different ways, including, for example, averaging V(t) over the required time interval; and calculating the length of path during the interval, and dividing by the length of the interval. Peristaltic frequency may be calculated, for example, by performing frequency analysis (FFT) on the velocity. Other parameter may also be determined.

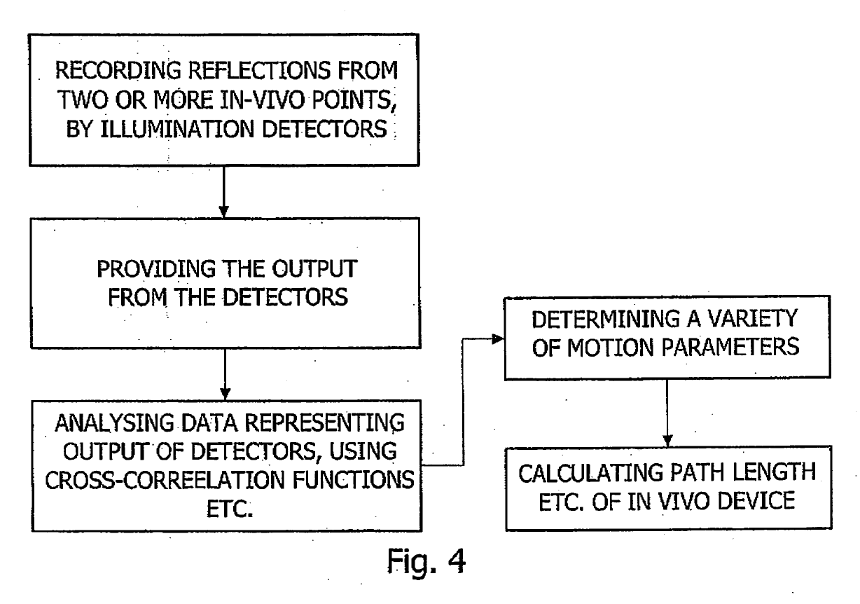

FIG. 4 is a flow chart depicting a series of operations of a method to determine motion parameters of an in-vivo device, according to an embodiment of the invention. As can be seen with reference to FIG. 4, reflections (or other local properties of the tissue, intestinal wall, etc.) of, for example, light energy, acoustic energy, electrical energy from in-vivo locations may be recorded in block 41 by one or more illumination detectors. The detectors may be for example illumination detectors, electrical impedance detectors, acoustic impedance detectors or any other suitable detectors. The output from the detectors may be provided in block 42, for example, illustrating the respective functions of the respective detectors, or the time required for one detector to move to the same internal location or point as another detector. The data representing the outputs of the respective detectors may be analyzed in block 43, for example, using cross-correlation functions. Other methods or functions of analysis may be used. Based on the time and the known (typically fixed) distance between the detectors, for example, various motion parameters, such as the velocity of in-vivo device movement, distance, instant and average velocity, frequency of peristaltic waves etc., may be calculated in block 44. The path length and other path characteristics, of in the vivo device, may also be calculated in block 45 based on relative movement of the detectors. Any combination of the above operations may be implemented. Further, other operations or series of operations may be used.

According to some embodiments of the present invention, a measurement counter 18, which may be used to measure points in time or periods of time, and which may also be used to activate or deactivate an in-vivo imaging device according to determined measurement parameters, may be located in, for example, reception unit 12, data processor 14, transmitter 125 or any other suitable location in in-vivo imaging system 100. Typically counter 18 may be functionally connected to at least one computer processing module or software module such as data processor 14 for processing and/or tacking calculations. The counter 18 may be reset manually at any suitable point, as with, for example, an odometer in a car, or may be reset automatically, such as by being triggered by events. For example, the counter 18 may be reset upon gastric emptying which may be, for example, detected by a pH sensor or any other suitable sensor (typically located on device 10) and determined by processor 14. Upon detection of such an event, the software or other process (e.g., a process being run on processor 14) may reset the counter 18, such that all distances calculated will start from a selected point, such as the duodenum. An automatic measurement operation, for example, may prevent path length measurement errors caused by in-vivo device tumbling or rotating that may occur in larger lumen such as, for example, the stomach. More than one memory or counter 18 may be used.

In accordance with an embodiment of the present invention, electromagnetic methods may be used for determination of motion parameters, by measuring electrical characteristics such as impedance or conductivity etc. For example, as known in the art momentary attenuation of the electromagnetic radiation by liquid can be measured so as to determine the momentary volume and velocity of the liquid. Similarly, embodiments of the present invention may utilize electrical properties to determine velocity of in-vivo device 10. Therefore electromagnetic radiation sensors may be used in place of or in addition to detectors 23 and 24, and whether these electromagnetic radiation sensors move relative to the irregularities of the lumen walls or cavities, or the irregularities move relative to the detectors (flow), the functions of the irregularities may be recorded. For example, the functions of the intestinal wall movement or of the momentary attenuation may be recorded.

Physiological tissues are typified by specific electrical impedance characteristics. Detectors 23 and 24 may be, for example, adapted to detect electric impedance from body lumen or cavities. Based on electrical measurements from detectors 23 and 24, processor 14 may determine, for example, particular in-vivo locations and enable calculation of motion parameters of an in-vivo imaging device relative to the determined particular in-vivo locations. Embodiments for the calculation of electrical characteristics of locations in a lumen using an in-vivo device are described, for example in U.S. Pat. No. 6,584,348 by the same assignee of the present invention, which is hereby incorporated by reference in its entirety.

Physiological tissues are typified by specific mechanical impedance characteristics. In accordance with an embodiment of the present invention, energy receiving units such as acoustic impedance measuring apparatus, such as ultrasonic transducers, detectors or other suitable acoustic entities may be provided, for example in motion parameter measurement unit 11. Acoustic impedance measuring apparatus may detect in-vivo locations based on acoustic impendence at particular locations. Motion parameter measurement unit 11 may include a plurality of acoustic impedance measuring apparatuses (e.g., detectors 23 and 24 may detect acoustical energy rather than light energy). For example, processor 14 may calculate local acoustic impedances based on ultrasonic waves reflected to and from locations in a lumen, to determine, for example, when a plurality of motion parameter detectors acquire data of a particular location. Upon establishment of a particular location of which data was acquired by two or more detectors, processor 14 may further calculate various motion parameters of in-vivo device 10. U.S. patent application Ser. No. 10/365,612, by the same assignee of the present invention, titled “DEVICE, SYSTEM AND METHOD FOR ACOUSTIC IN-VIVO MEASURING”, filed on 3 Feb. 2003, includes embodiment allowing for the detection of acoustic characteristics of locations in a lien using an in-vivo device.

According to an embodiment of the present invention, when, for example, electrical characteristics or acoustic characteristics of one location are similar to characteristics of another location, previously identified by appropriate measuring apparatus, as determined by, for example, processor 14, these locations may be defined as being a target location for measurement of in-vivo device 10 motion parameters. Processor 14 may calculate the time difference in detectors 23 and 24 reaching such a location. Since the distance between the locations of the two detectors on in-vivo device 10 is fixed, the distance traversed by in-vivo device 10 may be known, and processor 14 may calculate the velocity, direction of movement and various other movement parameters of in-vivo device in-vivo device 10.

According to some embodiments of the present invention, two-dimensional illumination detectors may be used. A two dimensional illumination detector, for example, may be located along the longitudinal axis of the in-vivo device 10, as well as the perpendicular axis. This embodiment, for example, may enable detection of longitudinal translocation of the in-vivo device 10, as well as movement of the in-vivo device 10 in the perpendicular direction (capsule rotation).

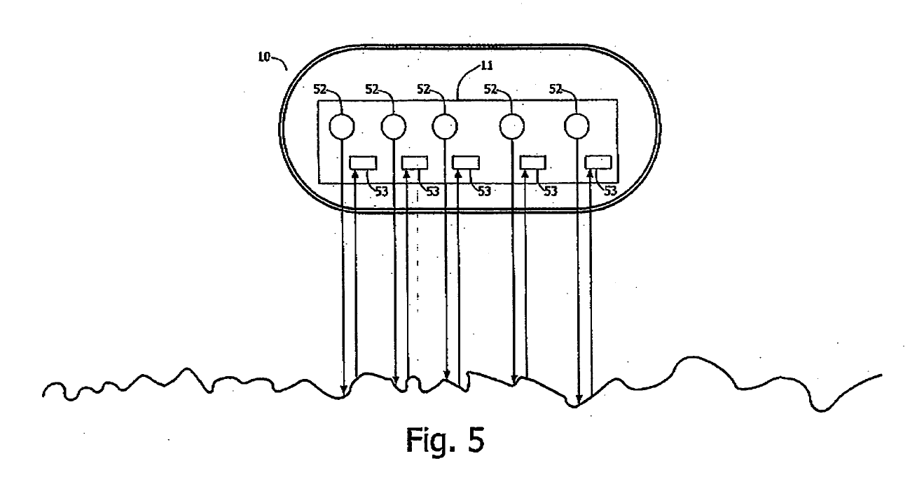

According to some embodiments of the present invention, a plurality of illuminators and/or detectors may be used, and the illuminators/detectors may be used in places other than as shown in FIG. 2A and FIG. 2B. As can be seen with reference to FIG. 5, a plurality of illumination detectors 53, or an array of detectors, may be used. A plurality of illumination sources 52 may be integrated into in-vivo imaging device 10. In this case, the motion parameters of the in-vivo device 10 may be calculated as an average motion from the data measured by the set (or a sub-set) of detectors. Alternative mathematical approaches may be applied. According to some embodiments of the present invention, a plurality of illumination sources and/or detectors may be placed in various locations on the circumference of in-vivo imaging device 10, thereby enabling identification of longitudinal movement and/or device 10 rotation. Such configurations of illumination sources and/or detectors may enable measurement of the longitudinal movement parameters and/or device 10 rotation parameters, for example, by using cross-correlations between pairs of the detectors.

According to some embodiments of the present invention, a method of calculating motion parameters may, for example, calculate the cross correlation function between several pairs of detectors. The method may calculate the corresponding Tij (time to get from detector i to detector j). The method may calculate, for example, Vij=Dij/Tij, where Vij is a velocity calculated based on the detectors i and j, and Dij is a distance between detectors i and j. According to some embodiments average velocity may be calculated by averaging various Vij data. Alternatively the most distinctive cross-correlation function from all the pairs of detectors may be taken. The most distinctive may include, for example, the function of having the maximal or minimal value. Two-dimensional detectors may be used. The above processes may be implemented in any combination.

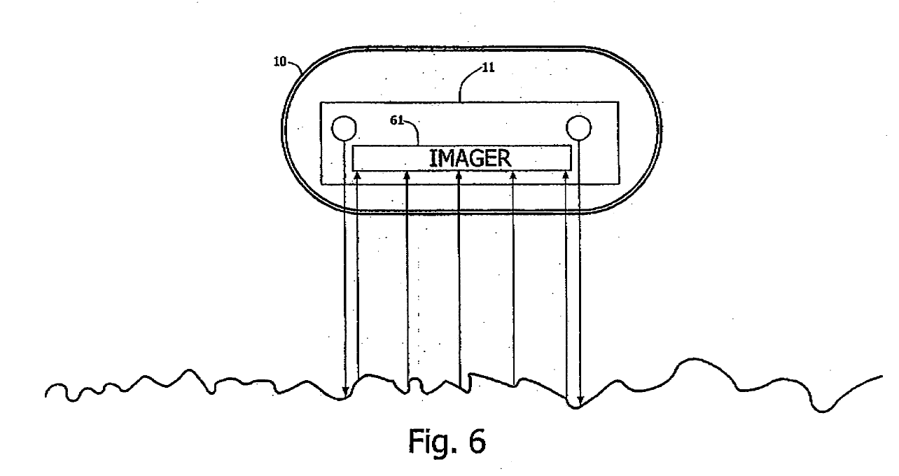

According to some embodiments of the present invention, a motion illumination imager 61 may be integrated into in-vivo imaging device 10. Motion illumination imager 61 may typically be located near an optical window, typically on at least one side of device 10, as can be seen with reference to FIG. 6, but optionally in other locations. Imager 61 may be, for example, an imager like imager 120 (FIG. 1) or any other suitable imaging apparatus that may feature a continuous imaging surface that may image multiple locations simultaneously. Imager 61 may, for example, be directly facing at least one side of in-vivo device 10, or may be otherwise positioned but may use a mirror, prism, etc. to view at least one side of device 10. For example, a forward-looking imager may be used, such that at least one side image may be sampled by applying mirrors, prisms and/or optical fibers etc. For example, the pixels of imager 61 may be considered as a set of motion illumination detectors 23 and 24. Imager 61 may function as a two dimensional array of detectors.

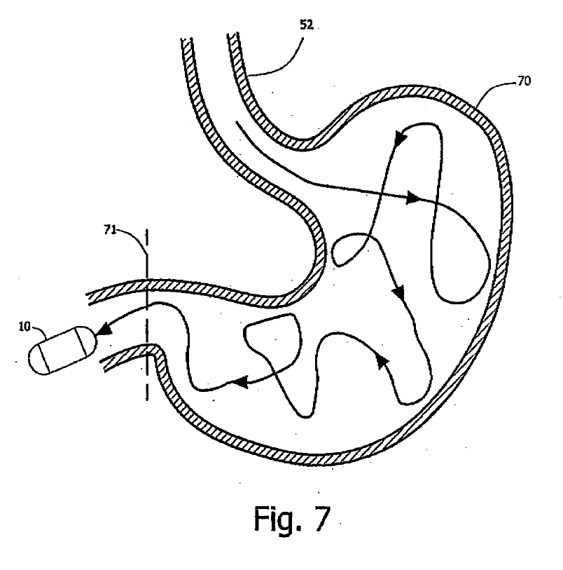

As can be seen with reference to FIG. 7, an estimated path 70 for an in-vivo device 10, such as an autonomous capsule, may be traced and displayed. The path 70 may represent the path traversed by an in-vivo device 10. As can be seen in FIG. 7, the output from displaying device indices a path traversed by in-vivo device 10 from a particular starting point, such as the duodenum, as indicated by feature 71, following a triggering event. According to some embodiments of the present invention, the path length 70 may be calculated and/or displayed, for example, by adding up the distances traveled by the in-vivo imaging device drug a selected period of time, or between selected points etc.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in illumination of the above teaching. It is, therefore, to be understood that the appended claims are intended to cover all such modifications and changes as fall within the true spirit of the invention.

What is claimed is:

1. An in-vivo imaging system comprising:
an in-vivo device including at least a plurality of illumination sources and an illumination detector, said illumination detector being connected to a processor,
said processor being configured to receive signals from the illumination detector and to calculate a rate of movement of the in-vivo device.

2. The system according to claim 1, wherein said processor is disposed within said in vivo device.

3. The system according to claim 1, wherein said processor is external to the in-vivo device

4. The system according to claim 1 comprising a transmitter.

5. The system according to claim 1 comprising a power source.

6. The system according to claim 1, comprising an imager.

7. The system according to claim 1, wherein said illumination detector is an imager.

8. The system according to claim 1, wherein said in-vivo device is autonomous.

9. The system according to claim 1, wherein said in-vivo device is a swallowable capsule.

10. A system for determining motion parameters for an in-vivo device, said system comprising:
an in-vivo device, said in-vivo device including at least a measurement unit for receiving signals reflected from a body lumen tissue and a transmitter; and
a reception unit; and
a processor unit.

11. The system according to claim 10, wherein the energy output units are selected from the group consisting of: illumination source; electrical current source; acoustic source.

12. A method for determining motion parameters of an in vivo device, the method comprising:
emitting signals from a plurality of energy output units disposed on an in-vivo device; and
recording reflections of the signals.

13. The method of claim 12, comprising calculating a distance moved by said in vivo device using the recorded reflections.

14. The method according to claim 12, wherein the energy units are illumination sources.

15. The method according to claim 12, wherein the energy units are selected from the group consisting of: electric current sources; acoustic energy sources.

16. The method according to claim 12 comprising comparing a first set of data of reflections from a first source to a second set of data of reflections from a second source.

17. The method according to claim 12, comprising analyzing reflection from a body lumen tissue using a cross-correlation function.

18. A measurement counter for activating or deactivating an in-vivo imaging device based on measurement parameters.

19. The measurement counter according to claim 18, wherein said measurement counter is located in an in vivo imaging device.

20. The measurement counter according to claim 18, wherein said measurement counter is connected to a processor.

21. A method comprising:
in an autonomous in-vivo device:
outputting an illumination signal;
recording a first reflected illumination signal; and
recording a second reflected illumination signal; and
determining, from the recorded signals, a movement of the in-vivo device.

22. The method of claim 21, comprising calculating a distance moved by said in vivo device using the recorded signals and a known distance between devices recording the reflected illumination signals.

23. The method of claim 21 comprising outputting illumination signals from multiple illumination sources.

24. The method of claim 21, wherein the determination is performed in the in-vivo device.

25. The method of claim 21, comprising comparing the recorded signals using a cross-correlation function.

26. The method of clam 21, comprising collecting images via the in-vivo device.
